(19) United States
(12) Patent Application Publication
(10) Pub. No.: US 2026/0175023 A1
(43) Pub. Date: Jun. 25, 2026

(54) DEVICES AND SYSTEMS OF INDUCING PAIN SENSATIONS IN VR ENVIRONMENTS THROUGH THERMAL GRILL ILLUSION (TGI)

(71) Applicant: City University of Hong Kong, Hong Kong (HK)

(72) Inventors: Kening ZHU, Hong Kong (HK); Haichen GAO, Hong Kong (HK); Shaoyu CAI, Singapore (SG)

(21) Appl. No.: 19/000,766

(22) Filed: Dec. 24, 2024

(51) Int. Cl. A61N 1/28; G06F 3/01; H10N 10/17

(52) U.S. Cl. CPC A61N 1/28; G06F 3/011; G06F 3/016; H10N 10/17

(57) ABSTRACT

A virtual reality (VR) system for generating pain sensations in VR environments using thermal grill illusion (TGI), comprising a VR device and a wearable thermos-haptic device with a thermoelectric stimulative module for inducing TGI-based pain sensations, a heat sink coupled to the module for temperature regulation and stabilization, and a thermocouple for monitoring and controlling temperature changes.

### FIELD OF THE INVENTION

The present invention generally relates to the visual simulation fields. More specifically the present invention relates to inducing pain sensations in VR environments to improve the sense of presence and body ownership.

### BACKGROUND OF THE INVENTION

With the rapid advancement of VR technology, researchers have developed various haptic devices to enable high-fidelity tactile experiences across different sensory modalities in virtual environments (Cai et al., 2024, 2020; Choi et al., 2017; Huang et al., 2023; Tanaka et al., 2023; Zhu et al., 2019a). Among these modalities, pain is recognized as an unpleasant sensory experience associated with potential or actual harm (Raja et al., 2020). Despite its discomfort, incorporating pain sensations into VR can enhance safety awareness in training simulations (Cervero, 2012) and enrich user experiences in entertainment, enlightenment, and social interactions (Benford et al., 2012; Fusaro et al., 2016).

Providing pain sensations in VR poses significant safety and ethical challenges. One cost-effective approach has been pseudo-haptics, where visual illusions evoke a perception of pain. For example, BurnAR (Weir et al., 2012) simulated the sensation of heating and burning through virtual flames in augmented reality (AR), while Clavelin et al. (Clavelin et al., 2023) manipulated pain perception during simulated finger dislocation. However, the effectiveness of pseudo-haptics varies among individuals, as some users may not experience vision-induced pain (Eckhoff et al., 2020). Moreover, relying solely on visual stimuli can lead to mismatched visual-haptic experiences, which may negatively impact user performance and immersion (Cai et al., 2024; Hochreiter et al., 2018).

Researchers have also explored haptic feedback to induce actual pain sensations. For instance, Impacto (Lopes et al., 2015) used electrical muscle stimulation (EMS) to replicate the sensation of hitting and being hit in VR. Similarly, Kono et al. (Kono et al., 2018) combined EMS with visual feedback to manipulate users' eyelids, inducing fear or pain. However, EMS-induced sensations are generally limited to instantaneous impacts rather than sustained pain. Other studies used Peltier devices, as demonstrated by Niijima et al. (Niijima et al., 2020), where users experienced heat or pain when sensitive body parts, such as lips or forearms, contacted the devices. However, exceeding the pain threshold with such methods risks actual skin damage.

Chemical stimulants have also been investigated for pain induction. Capsaicin, for example, can trigger pain and mechanical hyperalgesia on human skin (Simone et al., 1989). Douleur (Jiang et al., 2021) applied capsaicin to activate the trigeminal nerve, incorporating a Peltier module to adjust pain levels through heating or cooling. Lu et al. (Lu et al., 2021) studied painful sensations as the skin absorbed cinnamaldehyde. However, chemical stimulants are limited by latency in pain onset due to their dispersion process and the potential for skin irritation.

Compared to these methods, the thermal grill illusion (TGI) offers a safer, non-invasive way to induce pain sensations. TGI generates a paradoxical pain illusion by interlacing warm and cool stimuli within a non-harmful temperature range (Bach et al., 2011; Craig and Bushnell, 1994; Kern et al., 2008). Unlike chemical stimulants or EMS, TGI enables rapid and reversible pain induction on the skin's surface without causing harm. However, applying TGI in VR poses challenges, such as understanding how parameters like temperature-changing rates and stimulation locations affect TGI-induced pain perception, limiting its scalability in virtual environments.

To address these gaps, the present invention introduces a wearable thermal feedback device designed to create TGI-induced pain sensations in VR. By leveraging TGI, the device generates on-skin pain illusions without inflicting harm or requiring invasive procedures, providing a safer and more reliable solution for interactive VR scenarios. This innovation significantly improves the sense of presence and body ownership compared to VR experiences without haptic feedback, offering a novel approach to enhancing empathic, immersive, and interactive VR applications.

### SUMMARY OF THE INVENTION

It is an objective of the present invention to provide an apparatus, device, system, or method to solve the aforementioned technical problems.

In accordance with a first aspect of the present invention, a virtual reality (VR) system for inducing pain sensations in VR environments through thermal grill illusion (TGI) is present. Specifically, the system includes:
a VR device; and
a wearable thermos-haptic device, including:
   a thermoelectric stimulative module configured to evoke TGI-based pain sensations;
   a heat sink equipped with a cooling regulation module, and the heat sink is thermally coupled to one side of the thermoelectric stimulative module to regulate and stabilize the temperature change rate of the thermoelectric stimulative module; and
   a thermocouple configured to regulate and monitor the temperature changes of the thermoelectric stimulative module and the heat sink.

In accordance with one embodiment of the present invention, the thermoelectric stimulative module includes a warming element and a cooling element.

In accordance with another embodiment of the present invention, the warming element has a warming rate ranging from 1° C./s to 5° C./s; and the cooling element has a cooling rate ranging from 1° C./s to 5° C./s.

In accordance with one embodiment of the present invention, the warming and cooling rates is modulated to increase or decrease pain intensity.

In accordance with one embodiment of the present invention, a second side of the thermoelectric stimulative module directly contacts the skin of a user.

In accordance with one embodiment of the present invention, the warming element and the cooling element are separated by a gap of at least 2 mm to reduce thermal interference.

In accordance with one embodiment of the present invention, the gap is adjustable to create an expected pain intensity and pain area.

In accordance with one embodiment of the present invention, the system further includes a thermal insulation fabric covering the heat sink to prevent unintended heat transfer to the user's skin.

In accordance with one embodiment of the present invention, the cooling regulation module includes an inlet tube, an outlet tube and a reservoir, forming a closed-loop cooling circuit.

In accordance with one embodiment of the present invention, the system further includes an adjustable elastic band for secure attachment to various body parts and accommodating users of different sizes.

In accordance with one embodiment of the present invention, the system further includes a pressure sensor to ensure optimal contact force between the thermoelectric stimulative module and the user's skin.

In accordance with one embodiment of the present invention, the system further includes a microcontroller integrated with a proportional-integral-derivative (PID) control algorithm to regulate the temperature changes of the thermoelectric stimulative module.

In accordance with one embodiment of the present invention, the thermoelectric stimulative module provides customizable TGI stimuli patterns based on user input or preprogrammed VR scenarios.

In accordance with one embodiment of the present invention, the thermoelectric stimulative module is configured to synchronize with VR events to provide real-time haptic feedback.

In accordance with one embodiment of the present invention, the system further includes a data logging system to record user responses to TGI-based pain sensations for research or adaptive calibration purposes.

In accordance with one embodiment of the present invention, the actuation sequence of the warming element and cooling element is swapable to influence the spatial perception of pain, comprising directional sensations.

### BRIEF DESCRIPTION OF THE DRAWINGS

Embodiments of the invention are described in more details hereinafter with reference to the drawings, in which:

FIGS. 1A-1D depict a wearable thermos-haptic device in different views, in which FIG. 1A demonstrates the internal components and their arrangement, FIG. 1B shows the bottom view, FIG. 1C is a side view, and FIG. 1D is a schematic of a VR system incorporating a wearable thermos-haptic device in according to one embodiment of the present invention;

FIG. 2 depicts a system diagram in according to one embodiment of the present invention;

FIG. 3 depicts the step response of thermal stimuli with different temperature-changing rates and thermal directions;

FIGS. 4A-4B depict the configuration of the user study environment, in which FIG. 4A shows the user interface for evaluating pain intensity, coldness, and unpleasantness and FIG. 4B displays the setup environment where stimulus unit fully contacts the skin and halts upon a 2N increase in pressure as measured by the load cell;

FIGS. 5A-5E depicts the results of user perception, including user perception of perceived pain intensity on incipient moment (FIG. 5A) and 5-second past (FIG. 5B), coldness prior to the incipient moment of pain (FIG. 5C), unpleasantness (FIG. 5D), and detection time (FIG. 5E) regarding each temperature rates combination;

FIG. 6 depicts the adjustment of inter-stimulus distances (bottom-up view);

FIGS. 7A-7B depict a graphical user interface for participants to rate their perceived pain intensity, in which FIG. 7A is a pop-up window for drawing perceived pain area during stimulation period, featuring a pair of cross marks that indicate the approximate locations of the two stimulus units and FIG. 7B is a main window for evaluating generally perceived pain intensity and the directional movement of pain sensation;

FIG. 8 depicts the aggregated results of the pain areas drawn by all participants under the Sₙₒₙₑ condition;

FIGS. 9A-9E depict the results of user perception, in which FIG. 9A shows the user perceived average pain area relative to spacing interval, FIG. 9B shows the user perceived average pain area relative to temperature rates combination, FIG. 9C exhibits the overall pain intensity during stimulation period, FIG. 9D demonstrates the identification rate of the lateral motion direction of pain sensation relative to spacing interval, and FIG. 9E is the identification rate of the lateral motion direction of pain sensation relative to the stimulus activation sequences;

FIGS. 10A-10C depict the observed biosignals, in which FIG. 10A shows the change in heart rate is measured by comparing the average cardiac cycle before and after the stimulus in a 14-second window centered around each timestamp, FIG. 10B depicts the change in EDA values is measured within the same window by subtracting EDAbasal from EDAmax, and FIG. 10C shows a three-lead ECG placement and a two-lead EDA placement;

FIG. 11 depicts the questionnaire responses on the user experience; and

FIGS. 12A-12B depict the average variation of heart rate (FIG. 12A) and electrodermal response (FIG. 12B) in response to three stimulation modes.

### DETAILED DESCRIPTION

In the following description, devices and/or systems of for introducing pain sensations in VR environments through TGI and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, “virtual reality (VR)” is a simulated digital environment that immerses users in a three-dimensional, interactive experience through the use of computer-generated graphics, sounds, and sensory feedback. Unlike traditional screens or interfaces, VR allows users to interact with the virtual environment as though they are physically present within it. VR can simulate real-world scenarios or create entirely fictional settings, making it applicable in entertainment, education, healthcare, training, and many other fields.

A “VR device” is a system that enables users to access and interact with virtual reality environments. It typically consists of the following core components.

### Head-Mounted Display (HMD):

The primary interface for delivering visual and auditory VR content. It includes:
Screens: High-resolution displays, often with separate panels for each eye, to create stereoscopic 3D visuals.
Lenses: Adjust focal distances and provide an immersive field of view.
Motion Sensors: Include gyroscopes, accelerometers, and magnetometers for head tracking.

### Controllers:

Handheld devices that allow users to interact with the VR environment. Controllers are equipped with:
Buttons and Joysticks: For navigation and interaction.
Haptic Feedback: Provides tactile sensations to enhance realism.
Positional Tracking Sensors: To detect precise hand movements.

### Tracking System:

Tracks the user's position and movements within the VR space. This includes:
External Trackers or Cameras: Placed in the environment to monitor user movements.
Inside-Out Tracking: Uses cameras or sensors built into the HMD.

### Processing Unit:

The hardware responsible for rendering the VR environment and managing interactions. This can be:
Built-In: In standalone VR devices.
External: Using a PC or console connected to the VR headset.

### Audio System:

Integrated speakers or headphone connections to provide spatial and immersive sound.

### Input Devices (Optional):

Additional accessories, such as VR gloves, treadmills, or motion suits, to enhance interaction and simulate body movement in the virtual space.

### Power and Connectivity:

Includes batteries, USB-C connections, or wireless systems to ensure seamless operation and freedom of movement.

Modern VR devices combine these components to deliver high-quality, immersive experiences. They are increasingly being integrated with haptic feedback devices, eye-tracking technology, and AI-driven systems to improve interactivity and realism.

The thermal grill illusion (TGI), first identified in 1896, occurs when alternating warm and cool stimuli are applied to the skin, creating an illusory perception of intense heat and pain. This phenomenon is characterized by a unique prickling sensation that combines burning heat and pain. Studies indicate that the arrangement and number of alternating warm and cool stimuli units do not significantly impact the occurrence of TGI. Instead, factors such as a larger temperature range, which enhances pain intensity through sensory summation, and a larger thermal stimuli area, which correlates with increased pain perception, play a critical role. Harper et al. found that adaptation to the cool bars of the grill significantly reduced pain, suggesting that the sensation of coolness contributes to TGI-induced pain. Building on this, Hunter et al. observed that exposure to cool stimuli prior to applying the TGI could diminish pain sensations.

Despite extensive research on TGI and its impact on pain perception, certain aspects remain underexplored. For example, the influence of warming and cooling rates on TGI-induced pain and the integration of TGI with VR technology are not well understood. Furthermore, previous findings highlight a challenge in achieving strong simulated pain intensity due to the anticipatory cooling effect, which diminishes the pain sensation. This challenge could be mitigated by employing dynamic conditions that combine a rapid warming rate with a slower cooling rate, ensuring continuous temperature modulation while the skin is in contact with the grill.

The potential application of TGI-induced pain sensations in VR also remains largely unexplored. To address these gaps, the present invention investigates the effects of thermal feedback with varying signal configurations on TGI-induced pain sensations and explores its integration into interactive VR scenarios.

In accordance with a first aspect of the present invention, a VR system for inducing pain sensations in VR environments through TGI is provided. This system combines advanced VR technology with a wearable thermos-haptic device, enabling realistic pain sensations that enhance user immersion and interaction. The VR system includes a VR device integrated with a wearable thermos-haptic device comprising several specialized components.

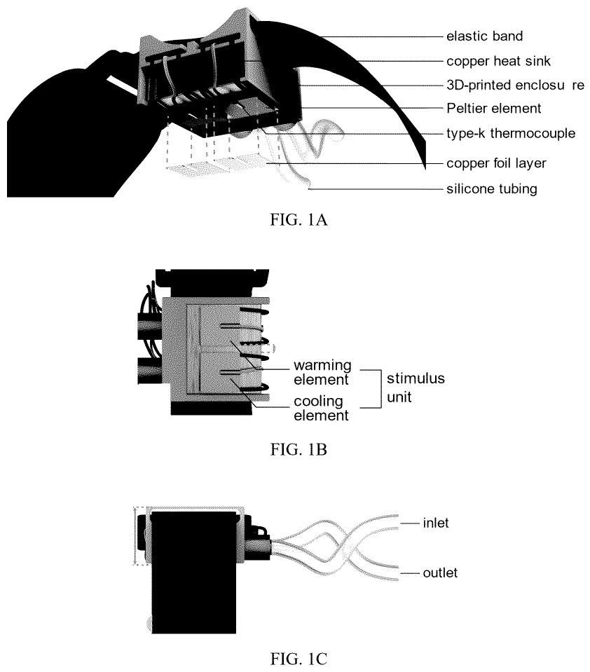

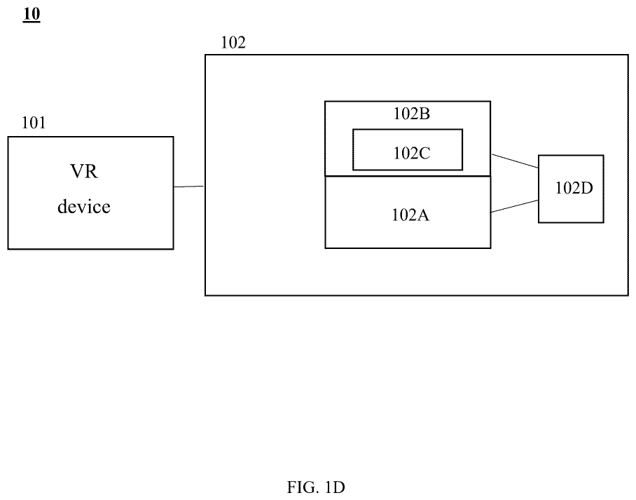

Referring to FIG. 1D, a VR system 10 in according to one embodiment of the present intention is depicted. The VR system 10 includes a VR device 101 and a wearable thermos-haptic device 102. Particularly, the wearable thermos-haptic device 102 has a thermoelectric stimulative module 102A for evoking TGI-based pain sensations, a heat sink 102B with a colling module 102C for regulating and stabilizing the temperature change rate of the thermoelectric stimulative module 102A; and a thermocouple 102D for regulating and monitoring the temperature changes of the thermoelectric stimulative module 102A and the heat sink 102B. It is worth noting that other thermal stimulation features could also be used as known in the art.

The wearable thermos-haptic device includes a thermoelectric stimulative module configured to evoke TGI-based pain sensations. This module achieves precise control over thermal stimuli through a heat sink thermally coupled to one side of the thermoelectric stimulative module. The heat sink is equipped with a cooling regulation module, ensuring stable and controlled temperature changes, thereby maintaining the consistency and safety of the TGI effect. Additionally, a thermocouple is employed to regulate and monitor temperature fluctuations in real time, enabling accurate control and dynamic adjustment of the pain-inducing stimuli.

The thermoelectric stimulative module is designed with both a warming element and a cooling element. These elements operate within specific thermal ranges, with the warming element providing temperature increases at rates from 1° C./s to 5° C./s and the cooling element facilitating temperature decreases at rates within the same range. The warming and cooling rates are modulated to adjust the intensity of the pain sensation, allowing customization of the haptic feedback to suit different VR scenarios or user preferences.

A key feature of the thermoelectric stimulative module is its direct contact with the user's skin on the opposite side, ensuring effective delivery of the thermal stimuli. To minimize thermal interference, the warming and cooling elements are separated by an adjustable gap of at least 2 mm. This gap can be modified to achieve desired pain intensities and adjust the perceived pain area, offering greater flexibility in the application of TGI. To further enhance user safety and comfort, the heat sink surface is covered with a thermal insulation fabric, preventing unintended heat transfer to the user's skin.

The cooling regulation module consists of an inlet tube, an outlet tube, and a reservoir, forming a closed-loop cooling circuit that ensures efficient thermal management. The wearable device also incorporates an adjustable elastic band, providing secure attachment to different body parts and accommodating a variety of user sizes. For optimal performance, a pressure sensor is integrated into the system to ensure appropriate contact force between the thermoelectric stimulative module and the user's skin.

The system is managed by a microcontroller equipped with a proportional-integral-derivative (PID) control algorithm. This algorithm facilitates precise temperature regulation and ensures consistent thermal transitions. Additionally, the thermoelectric stimulative module supports customizable TGI stimuli patterns, which can be tailored based on user input or preprogrammed VR scenarios.

The wearable thermos-haptic device synchronizes seamlessly with VR events, delivering real-time haptic feedback to align with the virtual environment's visual and auditory stimuli. This synchronization significantly enhances the sense of presence and immersion for users. Moreover, the system includes a data logging feature that records user responses to TGI-based pain sensations. These records can be utilized for research purposes or to adaptively calibrate the system for improved performance.

In addition, the actuation sequence of the warming and cooling elements can be swapped to influence the spatial perception of pain. This feature enables directional sensations, enriching the interactive VR experience by creating dynamic pain patterns that correspond to specific VR events.

Overall, the invention provides a versatile and innovative solution for integrating pain sensations into VR environments through the application of TGI. The described system enhances user immersion while maintaining safety, comfort, and adaptability, making it suitable for various applications, including entertainment, training, and therapeutic interventions.

### EXAMPLES

### Example 1. A Wearable Thermos-Haptic Device for VR Systems

As shown in FIG. 1A, the core component of the wearable thermos-haptic device is a Peltier-based temperature control module designed to induce the thermal grill illusion (TGI). The stimulative unit is encased within a 3D-printed enclosure and incorporates a copper heat sink. Beneath the heat sink, a pair of thermoelectric Peltier elements (12.5×12.5 mm, Module No.: HT16040) are positioned—one dedicated to warming and the other to cooling—to form an integrated stimulative unit. These elements are separated by a 2 mm gap, as shown in FIG. 1B, to minimize thermal interference between them. A Type-K thermocouple is attached for closed-loop temperature regulation. To prevent unintended heat transfer, the surface of the heat sink that may contact the user's skin is covered with thermal insulation fabric. To enhance user comfort, the device includes an adjustable elastic band for a secure fit, accommodating various body parts and user sizes. In this invention, the device is specifically designed for use on the forearm, as this region has been widely studied for TGI and exhibits an average level of thermal sensitivity and pain threshold compared to other body parts.

As shown in FIG. 1C, the temperature regulation system incorporates a water-cooling module for efficient thermal management. Silicone tubes connect the inflow and outflow ports of the copper heat sink to a water pump and a 370 mL reservoir, forming a closed water-cooling circuit. The reservoir, positioned above the water pump, serves as a repository for the cooling medium, which is circulated as needed. A Type-K thermocouple monitors the water temperature, maintaining it within a range of 23 to 25° C. (room temperature) for consistent operation during experiments.

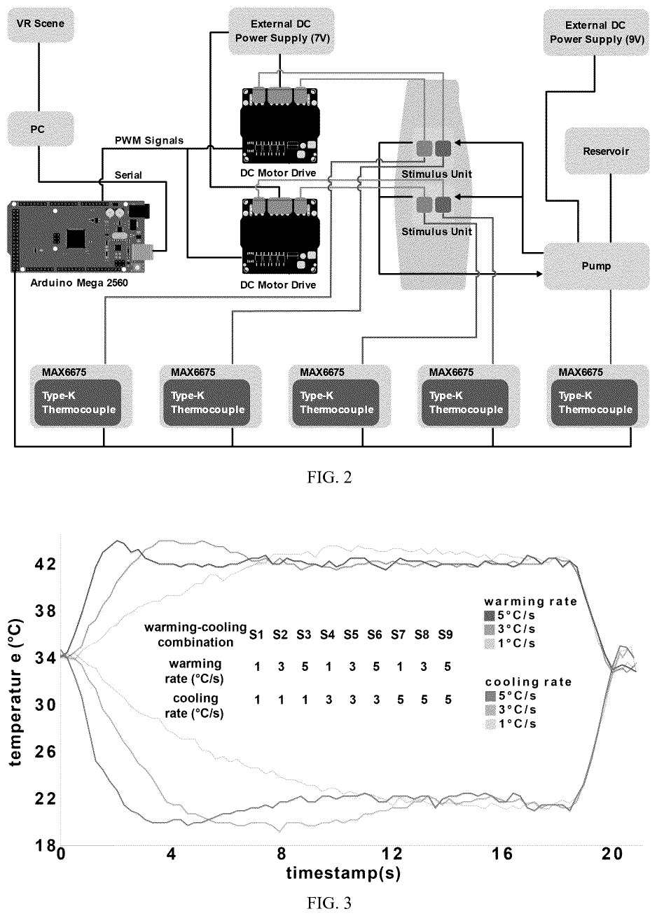

The system is controlled by an Arduino Mega 2560 microcontroller, which interfaces with two dual-channel motor drivers (L298N BTS7960) powered by an external 9V power supply to regulate the Peltier elements, as illustrated in FIG. 2. Real-time temperature monitoring is achieved through the Type-K thermocouples connected to MAX6675 modules, providing temperature readings at a sampling frequency of 4 Hz. These readings feed into a Proportional-Integral-Derivative (PID) algorithm, which adjusts pulse-width modulation (PWM) signals to achieve precise temperature control. This capability allows the system to deliver controlled thermal stimuli at various rates and directions, as depicted in FIG. 3.

### Example 2. User-Perception: The Perceived Intensity of Pain Sensations

This example examines how different warming and cooling rates influence users' perceived pain intensities through the wearable thermos-haptic device. The objective is to investigate the perception bandwidth of pain sensations enabled by the device. All experimental protocols, including this and subsequent investigations, were approved by the Ethics Committee of City University of Hong Kong (Approval No.: HU-STA-00000327).

A total of 12 participants (3 females and 9 males) with an average age of 28.7 years (SD=3.71) are recruited. The mean forearm skin temperature is 33.48° C. (SD=0.63), and the average forearm length is 23.25 cm (SD=2.22). All participants self-report as right-handed, free of physical impairments, and possessing normal thermal perception abilities.

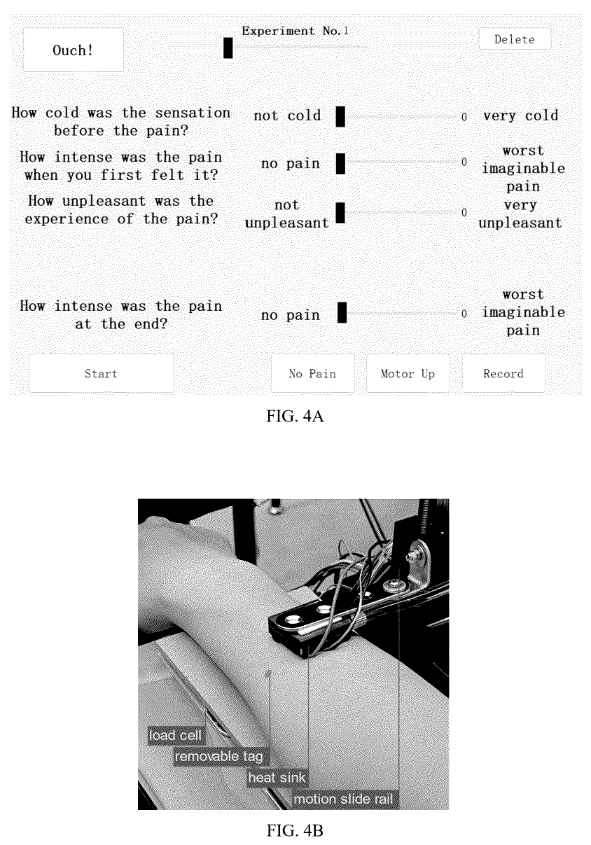

FIGS. 4A-4B depicts the configuration of the user study environment. Participants place their dominant forearms on a pressure-sensing surface equipped with load cells (GML670). A linear motion actuator (Module No.: 28T6*4-100) is used to control the perpendicular movement of Peltier actuators, ensuring consistent contact at the midpoint of the forearm with a constant applied normal force. Participants provide subjective ratings of the stimuli using a 12-inch touch-screen laptop operated with their non-dominant hands.

To minimize external sensory influences, a black cloth is placed between the participant's view and the stimulus unit to block visual cues. Additionally, participants are asked to wear noise-cancelling headphones to eliminate auditory distractions. The environmental conditions are regulated, with the ambient temperature maintained at 25° C. for consistency. This controlled setup ensures reliable data collection for evaluating the effects of thermal rate changes on pain perception.

A within-subjects design is adopted with the combination of warming and cooling rates as the independent variable. In this example, six temperature-changing rates are adopted, including three warming and three cooling. These temperature-changing configuration are chosen due to the clear distinguishability among them based on the results of the previous thermohaptic studies (Claus et al. 1987; Ken-shalo et al. 1968; Nakashige et al. 2009; Pertovaara and Kojo 1985; Wilson et al. 2011). This results in 3 cooling rates×3 warming rates=9 combinations of thermal stimuli for TGI induction, denoted as S1 to S9 shown in FIG. 3. A neutral starting temperature of 34° C. is chosen as this is within the defined ‘natural zone’ of thermal sensation. To ensure the participants' safety and comfort, the temperature range is constrained, with an upper limit of 42° C. and a lower limit of 22° C. which are below the thresholds for heat and cold pain. Each participant is subjected to a total of randomized 45 trials, with the 9 combinations of warming and cooling rates, each being repeated 5 times.

It is measured that participants' subjective ratings of perceived coldness prior to the onset of pain sensations, the unpleasantness and the perceived pain intensities at the incipient moment of pain perception, and the perceived pain intensities at five seconds after the detection of pain sensation. Participants are asked to provide subjective ratings on an 11-point Numerical Rating Scale (NRS) ranging from 0 to 10, assessing perceived coldness prior to pain onset, initial pain intensity, and unpleasantness associated with the pain. Additionally, the detection time is logged as the time interval between the initiation of the temperature shift and the point at which participants report the onset of pain sensation.

Each experiment involves one participant and one researcher. The researcher begins by explaining the study's purpose and procedures, obtaining the participant's consent, and measuring their skin temperature and dominant forearm length. The participant is seated at the experiment table, with their dominant forearm aligned at the central position marked on the load cell, corresponding to the midpoint of the stimulus unit. The central position is determined using the participant's previously measured forearm length.

Before the trials, participants undergo a training session to familiarize themselves with the experimental process and ensure they can perceive and tolerate the simulated pain illusion. During the training, participants experience two thermal stimuli: 1° C./s for cooling and warming, and 5° C./s for cooling and warming. These rates provide mild and intense TGI sensations, respectively, offering a range of thermal stimuli. Each trial begins when the participant presses the “START” button on the touchscreen (FIG. 4A). The linear motion actuator lowers the stimulus unit until a 2N increase in pressure is detected by the load cell, ensuring full contact and participant comfort. Once the 2N pressure is reached, the stimulus unit maintains a temperature of 34° C. for five seconds, accompanied by a visual countdown on the screen, before initiating the thermal stimulus.

During the stimulus, participants press the “OUCH” button as soon as they perceive pain and then rate the pain level, coldness, and unpleasantness using the touchscreen interface. The temperatures of the warming and cooling elements are simultaneously logged and maintained for five seconds. After this period, the screen displays “Trial Finish,” prompting participants to rate their current pain intensity. To conclude the trial, participants press the “MOTOR UP” and “RECORD” buttons, retracting the stimulus unit. A 15-second interval is provided between trials, allowing the skin temperature to return to baseline and alleviating any residual pain through self-touching of the stimulated area.

If participants do not report pain within 15 seconds of initiating the trial, they press the “NO PAIN” button and leave the rating sections blank. Participants are informed they may withdraw from the experiment at any time if the simulated pain sensation becomes intolerable. Notably, no participants chose to withdraw. Each experimental session lasted approximately 1 to 1.5 hours per participant, ensuring comprehensive data collection without causing undue discomfort.

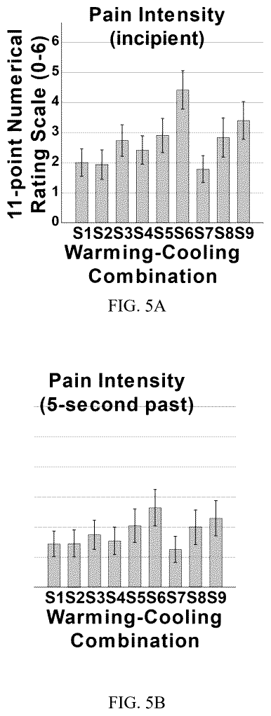

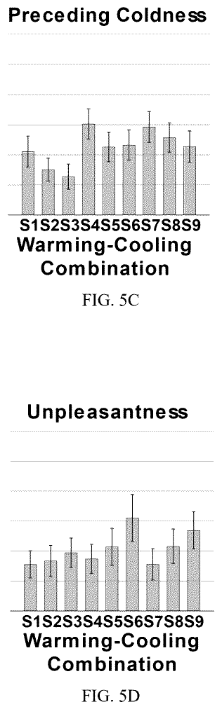

The effect of different warming and cooling rate combinations on perceived pain intensities at the incipient moment of pain perception is evaluated using a Friedman test (FIG. 5A). The analysis reveals that warming-cooling combinations significantly influence rated pain intensities (χ²(8)=92.583, p<0.001). A post-hoc pairwise Conover test further examines differences between each combination, with detailed results provided in Table 1.

TABLE 1
Details of pairwise comparison for incipient pain intensity (the “>”
indicates the significant difference for larger intensity level and
the “<” for lower level; * p ≤ 0.05; ** p ≤ 0.01; and *** p ≤ 0.001)

| Stimuli_1 | Relations | Stimuli_2 | p-value |
|---|---|---|---|
| S1 | < | S3 | * |
| | < | S5 | *** |
| | < | S6 | *** |
| | < | S8 | * |
| | < | S9 | *** |
| S2 | < | S3 | * |
| | < | S5 | *** |
| | < | S6 | **** |
| | < | S8 | * |
| | < | S9 | *** |
| S3 | < | S6 | * |
| | > | S7 | ** |
| | < | S9 | * |
| S4 | < | S6 | *** |
| | > | S7 | * |
| | < | S9 | ** |
| S5 | < | S6 | *** |
| | > | S7 | *** |
| S6 | > | S7 | *** |
| | > | S8 | *** |
| | > | S9 | * |
| S7 | < | S8 | ** |
| | < | S9 | *** |
| S8 | < | S9 | * |

Similarly, significant effects are observed for perceived pain intensities after five seconds of stimulation (χ²(8)=50.126, p<0.001), as shown in FIG. 5B. Pairwise comparisons using the Conover test are detailed in Table 2. A Wilcoxon signed-rank test compares perceived pain intensities at the initial moment and after five seconds (Table 3), revealing a significant reduction in all stimuli after five seconds, indicating a rapid decrease in pain sensations. Additionally, a Pearson correlation test shows a strong positive correlation between pain intensities at the initial moment and those after five seconds (r(538)=0.844, p<0.001).

TABLE 2
Details of pairwise comparison for 5-second post pain
intensity (the “>” indicates the significant
difference for larger intensity level and the “<”
for lower level; * p ≤ 0.05; ** p ≤ 0.01; and *** p ≤ 0.001)

| Stimuli_1 | Relations | Stimuli_2 | p-value |
|---|---|---|---|
| S1 | < | S5 | * |
| | < | S6 | *** |
| | < | S9 | ** |
| S2 | < | S5 | * |
| | < | S6 | *** |
| | < | S8 | * |
| | < | S9 | ** |
| S3 | < | S6 | *** |
| | > | S7 | * |
| S4 | < | S6 | *** |
| | < | S9 | ** |
| S5 | < | S6 | * |
| | > | S7 | ** |
| S6 | > | S7 | *** |
| | > | S8 | ** |
| S7 | < | S8 | ** |
| | < | S9 | *** |

TABLE 3
Details of Wilcoxon signed-rank comparison test for perceived pain
intensity on incipient moment (Intensity_1) and 5-second past
(Intensity_2) (the “>” indicates the significant difference
for larger intensity level; * p ≤ 0.05; ** p ≤ 0.01; and *** p ≤ 0.001)

| Intensity_1 | Relations | Intensity_2 | p-value | z-value |
|---|---|---|---|---|
| S1 | > | S1 | *** | 3.754 |
| S2 | > | S2 | *** | 3.467 |
| S3 | > | S3 | *** | 4.672 |
| S4 | > | S4 | *** | 4.967 |
| S5 | > | S5 | *** | 4.801 |
| S6 | > | S6 | *** | 6.275 |
| S7 | > | S7 | *** | 3.721 |
| S8 | > | S8 | *** | 4.108 |
| S9 | > | S9 | *** | 4.752 |

Subjective coldness and unpleasantness ratings are collected during the experiment (FIG. 5C and FIG. 5D). To achieve minimal cold sensation interference, the perceived coldness prior to pain onset is analyzed using a Friedman test, showing a significant effect (χ²(8)=67.747, p<0.001). Post-hoc pairwise Conover test results for coldness are detailed in Table 4.

TABLE 4
Details of pairwise comparison for coldness (the “>”
indicates the significant difference for larger intensity
level and the “<” for lower level; * p ≤
0.05; ** p ≤ 0.01; and *** p ≤ 0.001)

| Stimuli_1 | Relations | Stimuli_2 | p-value |
|---|---|---|---|
| S1 | > | S2 | * |
| | > | S3 | *** |
| | < | S4 | ** |
| | < | S7 | * |
| S2 | < | S4 | *** |
| | < | S5 | ** |
| | < | S6 | ** |
| | < | S7 | *** |
| | < | S8 | *** |
| | < | S9 | ** |
| S3 | < | S4 | *** |
| | < | S5 | *** |
| | < | S6 | *** |
| | < | S7 | *** |
| | < | S8 | *** |
| | < | S9 | * |
| S4 | > | S5 | * |
| | > | S6 | * |
| | > | S9 | * |
| S5 | < | S7 | * |
| S6 | < | S7 | * |
| S7 | > | S9 | * |

For unpleasantness ratings, a Friedman test reveals a significant main effect of warming-cooling combinations (χ²(8)=78.696, p<0.001). Post-hoc pairwise Conover test indicates that S6 (p<0.001) and S9 (p<0.01) elicit significantly higher unpleasantness ratings compared to other stimuli, with pairwise comparison results in Table 5. A Pearson correlation test confirms a strong positive association between initial pain intensity and unpleasantness ratings (r(538)=0.719, p<0.001).

TABLE 5
Details of pairwise comparison for unpleasantness (the “>”
indicates the significant difference for larger intensity level and
the “<” for lower level; * p ≤ 0.05; ** p ≤ 0.01; and *** p ≤ 0.001)

| Stimuli_1 | Relations | Stimuli_2 | p-value |
|---|---|---|---|
| S1 | < | S6 | *** |
| | < | S8 | * |
| | < | S9 | *** |
| S2 | < | S6 | *** |
| | < | S8 | * |
| | < | S9 | *** |
| S3 | < | S6 | *** |
| | | S7 | * |
| | > | S9 | *** |
| S4 | < | S6 | *** |
| | < | S9 | *** |
| S5 | < | S6 | *** |
| | > | S7 | * |
| | | S9 | *** |
| S6 | > | S7 | *** |
| | > | S8 | *** |
| S7 | < | S8 | ** |
| | < | S9 | *** |

Among the 540 trials, no pain perception is reported in 118 instances. The average response time for each stimulus is shown in FIG. 5E. For statistical analysis, response times for these trials are treated as 15 seconds. A repeated-measures ANOVA reveals a significant effect of warming-cooling combinations on response times (F(8, 88)=13.722, p<0.001), with post-hoc pairwise results in Table 6. Furthermore, a Pearson correlation test shows a negative correlation between detection time and initial pain intensity (r(538)=−0.768, p<0.001), suggesting that greater pain intensity leads to faster detection.

TABLE 6
Details of pairwise comparison for unpleasantness (the “>”
indicates the significant difference for larger intensity level and
the “<” for lower level; * p ≤ 0.05; ** p ≤ 0.01; and *** p ≤ 0.001)

| Stimuli_1 | Relations | Stimuli_2 | p-value |
|---|---|---|---|
| S1 | > | S3 | * |
| | > | S4 | * |
| | > | S5 | *** |
| | > | S5 | *** |
| | > | S9 | *** |
| S2 | > | S5 | *** |
| | > | S6 | *** |
| | > | S9 | *** |
| S3 | > | S6 | *** |
| S4 | > | S6 | *** |
| S5 | < | S7 | *** |
| S6 | < | S7 | *** |
| | < | S8 | *** |
| S7 | > | S9 | *** |
| S8 | > | S9 | ** |

In summary, the combination of warming and cooling rates significantly influences TGI-induced pain intensity ratings. A K-means clustering analysis divides the nine combinations into three clusters (Table 7), with significant differences in intensity ratings between clusters and no significant differences within clusters. Stimuli eliciting minimal cold sensations within their clusters (S2, S3, and S6) are selected for further experiments, indicating that the device can generate three distinct levels of pain illusion.

TABLE 7
Clustering nine temperature rates combinations into three
or four groups based on perceived pain intensity (results
remain consistent whether assessed at the incipient moment
of pain perception or five seconds thereafter)

| Temperature Rates Combination | | |
|---|---|---|
| (cooling rate-warming rate | Clusters (3) | Clusters (4) |
| S1 (1° C./s-1° C./s) | 2 | 2 |
| S2 (1° C./s-3° C./s) | 2 | 2 |
| S3 (1° C./s-5° C./s) | 3 | 3 |
| S4 (3° C./s-1° C./s) | 2 | 2 |
| S5 (3° C./s-3° C./s) | 3 | 3 |
| S6 (3° C./s-5° C./s) | 1 | 1 |
| S7 (5° C./s-1° C./s) | 2 | 2 |
| S8 (5° C./s-3° C./s) | 3 | 4 |
| S9 (5° C./s-5° C./s) | 3 | 3 |

This shows how dynamically changing thermal stimuli influence pain intensity perception. Results indicate that temperature-changing rate combinations significantly affect perceived pain intensity. Participants experience lower pain intensity with faster cooling rates, even when the total rate change remain identical, aligning with previous research suggesting that frequent cold sensations reduce pain in TGI contexts. The findings suggest that increasing the warm-to-cool rate ratio can augment pain intensity more efficiently than simply increasing the total rate change. Since cooling-stimulus Peltier devices generate excess heat, reducing dependency on cooling rates could enable more compact air-cooling systems, facilitating lightweight, untethered TGI devices. Additionally, a higher warm-to-cool ratio reduces preceding cold sensations, which is advantageous for scenarios requiring intense pain without prior coldness. However, scenarios like simulating sharp-edge touches can benefit from a higher cold-to-warm ratio for nuanced effects. Notably, 22.4% of trials lacks pain responses, mainly concentrates among three individuals, echoing prior findings of inherent insensitivity to TGI in some users.

### Example 3. User-Perception: The Perceived Area of Pain Sensations

This example investigates how thermal signals from two spatially distributed warming-cooling units on a user's forearm affect perceived pain intensity and the sensation's spatial area.

12 right-handed participants (9 males, 3 females) from a local university are recruited, adhering to the same criteria as the previous experiments (i.e., normal limb functionality and thermal sensation). To minimize potential biases or learning effects, none of the participants has participated in the earlier tests.

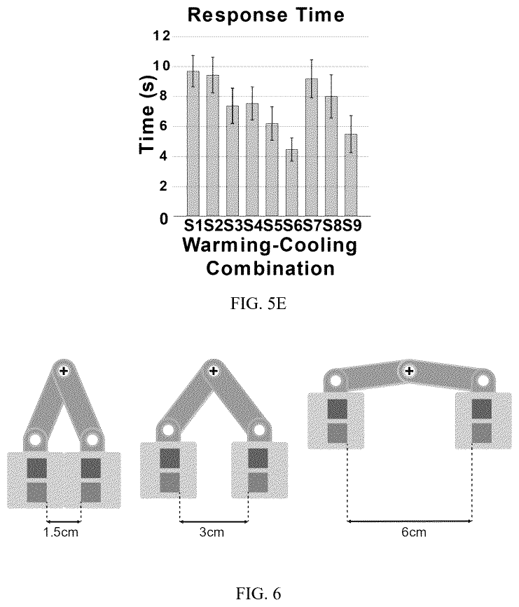

For this experiment, an additional stimulus unit is affixed alongside the original unit on the end effector of the linear motion actuator. To allow for adjustable inter-unit distances, each unit is mounted on the end of pin-jointed beams, as illustrated in FIG. 6.

A within-subject design is adopted, with three independent variables: the distance between the two stimulus units, the combination of warming and cooling rates, and the actuation sequence of the stimulus units. Considering the two-point discrimination (2PD) threshold for pain sensation on the forearm, distances of 1.5 cm, 3 cm, and 6 cm are selected. The two stimulus units are symmetrically aligned relative to the forearm's midpoint. Stimuli combinations S2, S3, and S6 (as previously selected) are applied, along with three actuation sequences: distal unit activation followed by proximal unit activation (denoted as S_{down}), proximal unit activation followed by distal unit activation (denoted as Sᵤₚ), and simultaneous activation of both units (denoted as Sₙₒₙₑ).

The stimulus durations are detailed in Table 8. For Sᵤₚ and S_{down}, the subsequent stimulus is initiated once the prior unit achieves its maximum temperature differential of 20° C. (between 42° C. and 22° C.), while the prior stimulus maintains this maximum differential. The trial ends when the subsequent unit also reaches its maximum differential. For Sₙₒₙₑ, both units are activated simultaneously and stopped when both achieved the maximum temperature differential.

TABLE 8
The duration of each stimulus

| Temperature Rates Combination | Actuation | Stimulation |
|---|---|---|
| (cooling rate-warming rate | Sequence | Duration (s) |
| S2 (1° C./s-3° C./s) | Sₙₒₙₑ | 18 |
| | Sᵤₚ | 24 |
| | S_{down} | 24 |
| S3 (1° C./s-3° C./s) | Sₙₒₙₑ | 18 |
| | Sᵤₚ | 24 |
| | S_{down} | 24 |
| S6 (3° C./s-5° C./s) | Sₙₒₙₑ | 6 |
| | Sᵤₚ | 8 |
| | S_{down} | 8 |

Each participant completes a total of 81 trials (3 distances×3 warming-cooling combinations×3 actuation sequences×3 repetitions). Distance presentation order is counterbalanced using a Latin square design, while stimulus presentations within each block are randomized. Dependent variables include participants' subjective ratings of perceived pain intensity, accuracy in identifying the lateral motion direction of the pain sensation, and the pixel count representing the user-drawn pain area.

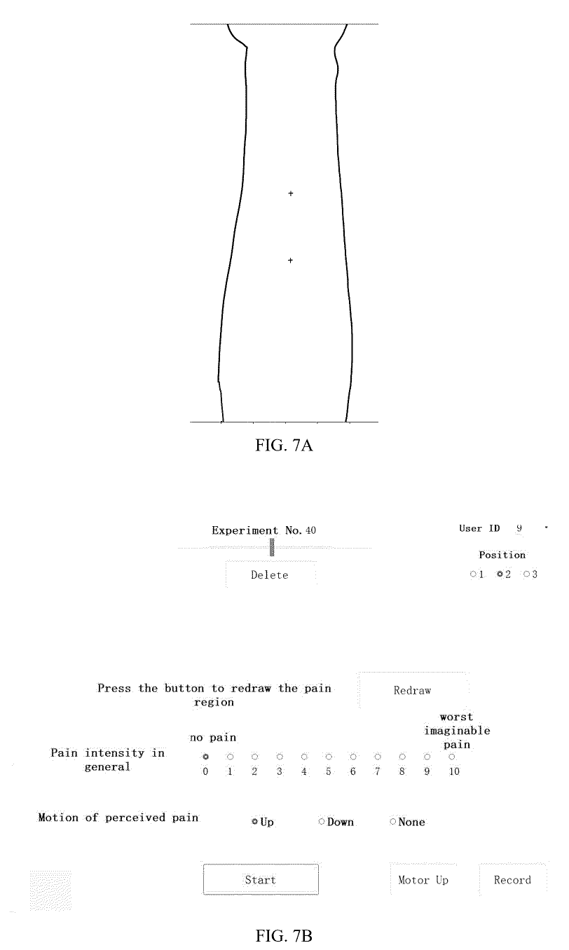

A structured experimental procedure consisting of “Introduction-Pre-Questionnaire-Training-Testing” is adopted. During the training session, participants are exposed to randomized stimuli under a fixed inter-unit distance of 3 cm. Participants repeat the trials until they self-report familiarity with the experimental procedure. After each trial, a graphical user interface (FIGS. 7A-7B) prompts participants to rate their perceived pain intensity on a scale of 0 to 10, indicate the lateral direction of the pain (DOWN, UP, or NONE), and draw the perceived pain area.

A 15-second break is provided between consecutive trials, with an extended 5-minute break after each inter-unit distance condition. The total duration of the experiment is approximately 2 to 2.5 hours per participant.

The perceived pain area is quantified by counting the pixels of the participant-drawn image. A multi-factorial repeated-measures ANOVA reveals a significant effect of inter-unit distance on the size of the perceived pain area

$$( F ( 2 , 22 ) = 16.252 , p < 0.001 , {η}_{p}^{2} = 0.317 ) .$$

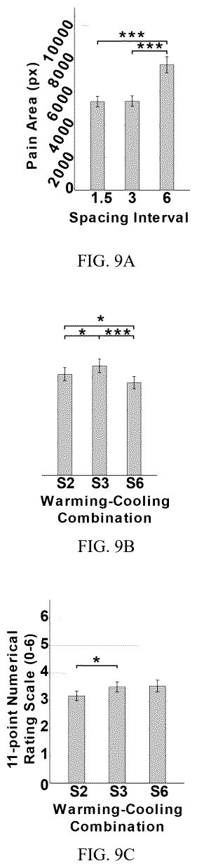

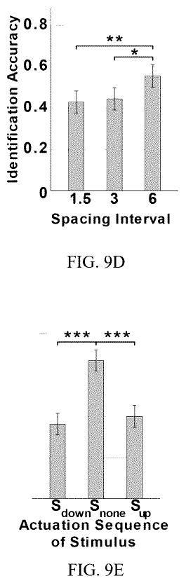

Post-hoe pairwise comparisons (FIG. 9A) indicate that a 6 cm distance results in significantly larger pain areas compared to 3 cm and 1.5 cm distances (p<0.001). Additionally, warming-cooling combinations have a significant impact on perceived pain are:

$$( F ( 2 , 22 ) = 14.9 \; 99 , p < 0.0 \; 01 , {η}_{p}^{2} = 0.3 ) .$$

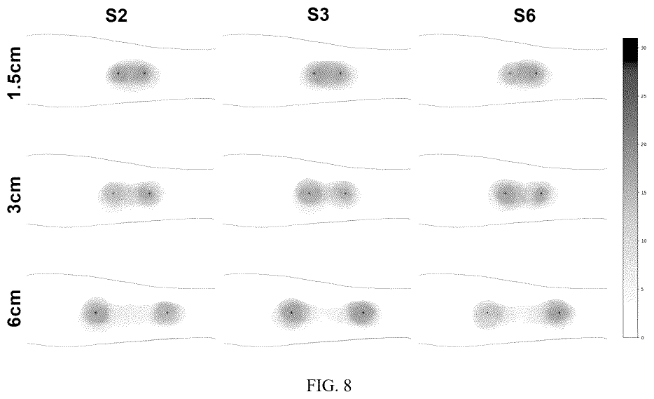

Post-hoc analysis (FIG. 9B) reveals that S3 elicits a significantly larger pain area than S2 (p=0.025) and S6 (p<0.001). Furthermore, S2 induces a significantly more expansive pain area than S6 (p=0.022). However, no significant effect of actuation sequences is observed. Heat maps of participant-drawn pain areas for different warming-cooling combinations and inter-unit distances, under simultaneous activation (Sₙₒₙₑ), are shown in FIG. 8.

A multi-factorial repeated-measures ANOVA with Aligned Rank Transform (ART) reveals a significant main effect of warming and cooling rate combinations on general pain intensity ratings

$$( F ( 2 , 22 ) = 4 , p = 0.0 \; 23 , {η}_{p}^{2} = 0.103 ) .$$

Post-hoc pairwise comparisons show that S3 elicits significantly higher pain intensity than S2 (p=0.034). S6 yields marginally higher pain intensity than S2 (p=0.054), but no significant difference is found between S6 and S3 (FIG. 9C). Neither inter-unit distance nor actuation sequence significantly affects perceived pain intensity ratings.

As shown in FIG. 9D and FIG. 9E, participants show low accuracy in identifying the lateral motion direction of the illusory pain, with mean accuracy rates of 42.90% (SD=23.66%) for 1.5 cm, 44.45% (SD=18.37%) for 3 cm, and 55.56% (SD=10.76%) for 6 cm. For 1.5 cm and 3 cm distances, most trials are perceived as non-directional stimuli. However, identification accuracy improves with the inter-unit distance of 6 cm, as shown in FIG. 9D. This improvement may be attributed to the inherently low spatial acuity of thermal perception.

This experiment shows how spatial parameters (e.g., inter-unit distance, temperature-changing rate, and actuation sequence) influence perceived pain area and intensity. Increasing the inter-unit distance from 3 cm to 6 cm significantly expands the perceived pain area. Regarding temperature-changing rates, S3 induces the largest pain area and strongest intensity, though no significant intensity difference is observed between S3 and S6. Compared to the Example 2, fewer pain-free trials occur, particularly none for S2 and S3, and only 2.8% for Sₙₒₙₑ at a 1.5 cm distance. Larger stimulation areas enhance TGI perception, especially for mild pain sensations. Participants also identified lateral pain motion more accurately at 6 cm spacing (˜70%), though the overall accuracy remained low, potentially due to insufficient distance for clear lateral motion detection. These findings indicate potential for integrating TGI-triggering arrays into wearable devices for dynamic pain patterns in VR.

### Example 4. User-Perception: User Experience with the System in VR Environments

A user study is conducted to evaluate the integration of the TGI system within VR environments and its influence on users' sense of presence, body ownership, and emotional states, as reflected in biosignals during immersive VR experiences.

Twelve participants (8 males, 4 females) are recruited for this experiment, with an average age of 26.4 years (SD=3.83). All participants are right-handed, possessed normal limb functionality, and have no involvement in previous experiments. While all participants have prior experience with VR, only two have encountered haptic feedback applied to the skin in a VR context.

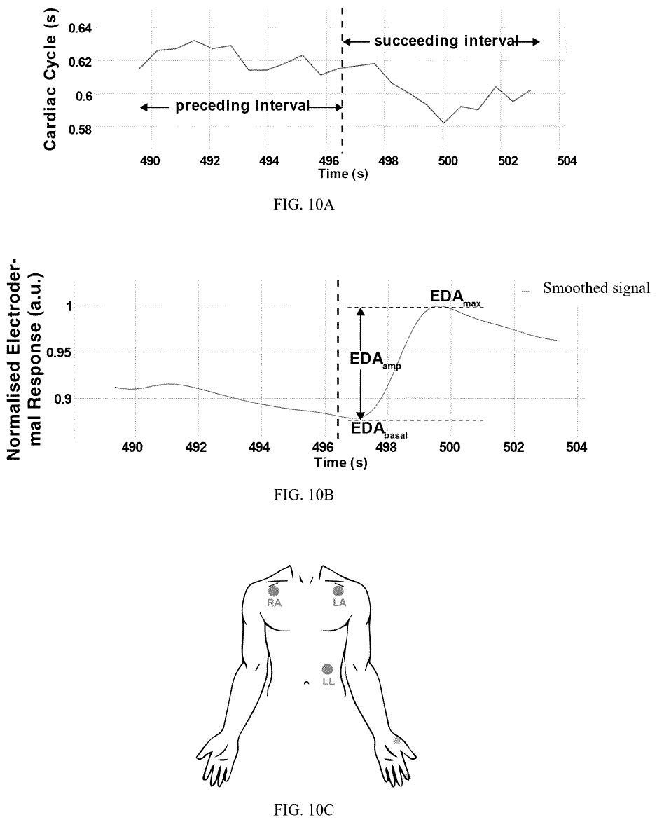

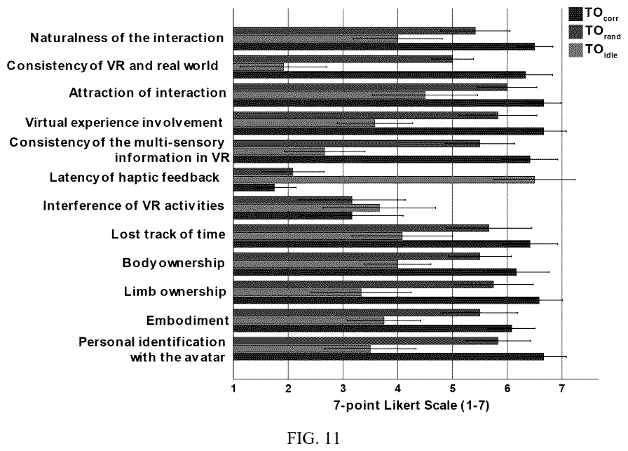

The TGI system is deployed to provide haptic feedback on the participants' forearms. A custom VR application including two distinct scenes is developed using the Unity3D engine (version 2020.3.40f1). The setup includes an HTC Vive Pro HMD and a pair of HTC Vive handheld controllers for virtual object interaction. Biosignal data—heart rate (HR), electrocardiogramand electrodermal activity (EDA)—are collected using the BlTalino toolkit. Electrodes are adhered to recommended positions based on existing literature, as shown in FIG. 10C, to ensure precise biosignal measurement while minimizing signal interference during VR activities. The collected biosignal data are continuously monitored and archived using the OpenSignals software on a PC.

The experiment follows a within-subjects design with three VR conditions: (1) wearing the device with temperature maintained at an idle state (34° C., denoted as TO_{idle}), (2) wearing the device with TGI-based haptic stimuli synchronized to the VR visual content (denoted as TO_{corr}), and (3) wearing the device with randomized, non-correlated TGI-based haptic stimuli (denoted as TO_{rand}). The inter-unit distances of 6 cm and 1.5 cm are used, and combined with S2, S3, and S6 stimuli, resulting in six distinct stimuli profiles.

To maintain the sense of presence in VR and minimize disruptions to immersion caused by mid-experiment changes in inter-stimulus spacing, two separate VR scenes are designed: a jungle navigation scene and a dragon-fighting scenario. In the jungle scene, participants are tasked with navigating a stone path to reach a campfire. In the dragon-fighting scene, participants defend themselves against a dragon and retaliated using a bow and arrow. Pain-triggering events in both scenes are tailored to align with the specific profiles of the corresponding stimuli, as outlined in Table 9.

TABLE 9
Six events and their corresponding stimulus features

| | | Warming-cooling combination | Distance |
|---|---|---|---|
| Scene | Event | (intensity-area) | (spot-area) |
| Jungle | Poked by pointed leaf | S6 (acute-small) | 1.5 (single-small) |
| navigation | Scratched by serrated leaf | S3 (acute-large) | 1.5 (single-small) |
| | Applying medicine to the wound | S2 (mild-medium) | 1.5 (single-small) |
| Against the | Defending fireball strike with an | S6 (acute-small) | 6 (multi-large) |
| dragon | arm shield | | |
| | Blocking falling rocks | S3 (acute-large) | 6 (multi-large) |
| | Triggering old injury while | S2 (mild-medium) | 6 (multi-large) |
| | drawing | | |

The dependent variables in this experiment include participants' sense of presence and body ownership within the VR environment, assessed through a haptic-focused questionnaire. Additionally, variations in biosignals before and after stimuli are analyzed to evaluate participants' real-time physiological responses during interactions within the VR scenarios.

During the experiment, participants are introduced to the study objectives and outfitted with the device, VR HMD, and biosignal sensors under the researcher's guidance. Participants are trained to interact with the virtual environment using handheld controllers. The study consists of three testing conditions, counterbalanced via a Latin square design: TO_{idle} (device maintained at 34° C. idle state), TO_{corr} (TGI-based haptic stimuli synchronized with VR visual content), and TO_{rand} (randomized TGI-based stimuli with no correlation to VR content). Within each condition, the sequence of the two VR scenes (jungle navigation and dragon-fighting) is randomized. During VR interactions, pain-triggering events are annotated with timestamps in the OpenSignals software. After each sub-session, participants remove the HMD and complete a touchscreen questionnaire using a 7-point Likert scale. The session concludes with a semi-structured interview to explore participants' experiences in depth. Each experiment lasted approximately 45 minutes per participant.

Participant ratings of presence and body ownership within VR are shown in FIG. 11. A Friedman test reveals that stimulation mode significantly influences several dimensions: naturalness of interaction (χ²(2)=19.581, p<0.001), consistency of VR and real world (χ²(2)=22.136, p<0.001), attraction of interaction (χ²(2)=15.548, p<0.001), experience involvement (χ²(2)=20.333, p<0.001), multi-sensory consistency (χ²(2)=21.565, p<0.001), latency of haptic feedback (χ²(2)=21.333, p<0.001), and time distortion (χ²(2)=18.865, p<0.001). Post-hoc Conover's test shows that TO_{corr} and TO_{rand} outperform TO_{idle} across most dimensions except latency. Additionally, TO_{corr} marginally outperforms TO_{rand} in naturalness (p=0.051) and consistency of VR and real world (p=0.054).

For body ownership, Friedman tests reveal significant effects on ownership (χ²(2)=16.233, p<0.001), limb ownership (χ²(2)=21.273, p<0.001), embodiment (χ²(2)=18.878, p<0.001), and identification with the avatar (χ²(2)=21.571, p<0.001). Post-hoc Conover's tests confirm that TO_{corr} and TO_{rand} are superior to TO_{idle} across all dimensions, with no significant differences between TO_{corr} and TO_{rand}.

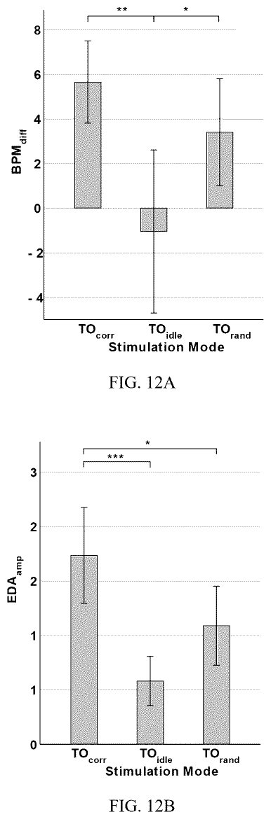

Changes in heart rate (HR) and EDA are observed in response to stimuli, as shown in FIGS. 10A and 10B. HR data (FIG. 12A) and EDA data (FIG. 12B) within 7 seconds before and after each event are compared. Repeated-measures ANOVA reveals a significant effect of stimulation mode on HK variation

$$( F ( 2 , 18 ) = 7.142 , p = 0.005 , {η}_{p}^{2} = 0.442 ) .$$

Post-hoc tests show significant HR increases under TO_{corr} (p=0.005) and TO_{rand} (p=0.048) compared to TO_{idle}. No interaction between stimulation mode and event type is detected, indicating consistent HR changes across VR events within each condition.

For EDAamp signals, a 0-35 Hz bandpass filter is applied to remove high-frequency noise. Data from four participants are excluded due to flat plateau signals, likely caused by sweating. Repeated-measures ANOVA reveals a significant effect of stimulation mode on EDAamp values

$$( F ( 2 , 14 ) = 11.5 \; 93 , p = 0.0 \; 01 , {η}_{p}^{2} = 0.623 ) .$$

Post-hoc tests indicate significantly higher EDAamp values under TO_{corr} compared to TO_{rand} (p=0.027) and TO_{idle} (p<0.001).

Participants are interviewed after the experiment to gather feedback. Compared to the condition without haptic feedback (TO_{idle}), both pain-inducing conditions (TO_{corr} and TO_{rand}) enhance the sense of presence and immersion in the virtual environment. However, most participants cannot definitively identify a superior experience between TO_{corr} and TO_{rand}. For instance, P10 remarkes, “I may notice the discrepancy of pain areas if I focus on it.” P8 notes that the somatosensory localization of pain, such as being cut by a leaf in TO_{corr}, aligns better with the visual cues, and the pain intensity from the cut matches his expectations better than applying medicine to the wound. Six participants highlight the realism of using a shield to block fireballs, with P9 stating, “The rendered stimulus spreads out like the impact of being struck, which felt very authentic.” Regarding haptic feedback latency, 10 of the 12 participants report perceiving little to no delay. The remaining two notice slight latency but find it inconsequential. P3 comments, “It's not bad, especially for virtual environments, because your focus is on other things.”

By integrating the device into VR, its impact on user experience is assessed, including sense of presence, ownership, and stress levels. Both TO_{corr} and TO_{rand} significantly enhance presence and ownership compared to TO_{idle}, with TO_{corr} slightly outperforming TO_{rand} in fostering natural interaction and aligning VR with real-world experiences. Even when not perfectly align with visual content, TGI-based pain illusions foster a strong sense of presence and ownership in VR. Participants' sense of presence ratings reflects subjective expectations of pain-triggering events in VR, such as blocking falling rocks or fireballs. As these scenarios lack real-world analogs, users' ratings may lack a “ground truth,” explaining the similarity between TO_{corr} and TO_{rand} ratings. Physiological responses indicate that TO_{corr} elicits higher stress levels, reflects in increased skin conductance response (SCR), correlating with enhanced fear learning. This suggests potential applications in safety-related VR training and education, such as entertainment (e.g., VR gaming and 4D cinema) to enhance immersion, medical education for training healthcare professionals in pain management and empathy, and hazardous scenario training (e.g., firefighting, military operations) to improve skill acquisition.

In summary, the present invention introduces a wearable thermos-haptic device that leverages the TGI to simulate pain sensations in VR without causing actual invasive/non-invasive harm. The results of the user-perception experiments reveal that higher temperature-changing rates, particularly with increased warming, are associated with more intense pain perceived by the participants through the device. Furthermore, a higher ratio of warm-to-cool temperature transitions reduces the sensation of coldness prior to pain. The experiments also show that introducing an additional stimulus unit potentially heightens pain perception, and altering the spacing between stimulus units modifies the perceived pain area. Lastly, the user study in VR demonstrates that the device significantly enhances the sense of presence and body ownership for the participants, as well as elevated their biosignal-indicated arousal levels.

The terms “a” and “an” used herein are intended to be understood as meaning one or more unless explicitly stated otherwise. Moreover, the terms “first”, “second”, “third”, etc. are used merely as labels, and are not intended to impose numerical requirements on or to establish a certain ranking of importance of their objects.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A virtual reality (VR) system for inducing pain sensations in VR environments through thermal grill illusion (TGI), comprising:
a VR device; and
a wearable thermos-haptic device, comprising:
a thermoelectric stimulative module configured to evoke TGI-based pain sensations;
a heat sink equipped with a cooling regulation module, wherein the heat sink is thermally coupled to one side of the thermoelectric stimulative module to regulate and stabilize the temperature change rate of the thermoelectric stimulative module; and
a thermocouple configured to regulate and monitor the temperature changes of the thermoelectric stimulative module and the heat sink.

2. The VR system of claim 1, wherein the thermoelectric stimulative module comprises a warming element and a cooling element.

3. The VR system of claim 2, wherein the warming element has a warming rate ranging from 1° C./s to 5° C./s; and the cooling element has a cooling rate ranging from 1° C./s to 5° C./s.

4. The VR system of claim 2, wherein the warming and cooling rates is modulated to increase or decrease pain intensity.

5. The VR system of claim 1, wherein a second side of the thermoelectric stimulative module directly contacts the skin of a user.

6. The VR system of claim 2, wherein the warming element and the cooling element are separated by a gap of at least 2 mm to reduce thermal interference.

7. The VR system of claim 6, wherein the gap is adjustable to create an expected pain intensity and pain area.

8. The VR system of claim 5, further comprising a thermal insulation fabric covering the heat sink to prevent unintended heat transfer to the user's skin.

9. The VR system of claim 1, wherein the cooling regulation module comprises an inlet tube, an outlet tube and a reservoir, forming a closed-loop cooling circuit.

10. The VR system of claim 1, further comprising an adjustable elastic band for secure attachment to various body parts and accommodating users of different sizes.

11. The VR system of claim 5, further comprising a pressure sensor to ensure optimal contact force between the thermoelectric stimulative module and the user's skin.

12. The VR system of claim 1, further comprising a microcontroller integrated with a proportional-integral-derivative (PID) control algorithm to regulate the temperature changes of the thermoelectric stimulative module.

13. The VR system of claim 10, wherein the thermoelectric stimulative module provides customizable TGI stimuli patterns based on user input or preprogrammed VR scenarios.

14. The VR system of claim 1, wherein the thermoelectric stimulative module is configured to synchronize with VR events to provide real-time haptic feedback.

15. The VR system of claim 1, further comprising a data logging system to record user responses to TGI-based pain sensations for research or adaptive calibration purposes.

16. The VR system of claim 2, wherein the actuation sequence of the warming element and cooling element is swapable to influence the spatial perception of pain, comprising directional sensations.
